(19) United States
(12) Patent Application Publication
(10) Pub. No.: US 2026/0335553 A1
(43) Pub. Date: Oct. 29, 2026

(54) DRUG DELIVERY DEVICE AND METHOD OF MIXING

(71) Applicant: Oval Medical Technologies Limited, Cambridge, Cambridgeshire (GB)

(72) Inventor: Matthew YOUNG, Cambridge, Cambridgeshire (GB)

(21) Appl. No.: 19/122,412

(22) PCT Filed: Oct. 19, 2023
(86) PCT No.: PCT/GB2023/052717

§ 371 Date: Apr. 18, 2025

(30) Foreign Application Priority Data
Oct. 19, 2022 (GB) 2215446.2

(51) Int. Cl. A61M 5/20

(52) U.S. Cl. CPC A61M 5/2033; A61M 5/206; A61M 2005/202

(57) ABSTRACT

There is provided a drug delivery device comprising a drug container assembly including a drug container containing a drug, a needle, a plunger rod, a first stored energy source, a needle insertion stored energy source, and an activation means. Movement of the activation means releases the needle insertion stored energy source to move the needle from a needle pre-insertion position to a needle insertion position and releases the first stored energy source to move the plunger rod towards and relative to the drug container assembly such that the drug container assembly applies a force to the drug before the needle reaches the needle insertion position. A method of mixing a drug is also provided.

### FIELD OF INVENTION

The present invention relates to a drug delivery device such as an autoinjector, and to a method of mixing a drug in a drug delivery device.

### BACKGROUND

Autoinjectors may automate one or both of needle insertion and drug delivery. Early autoinjector designs tended to have a single spring which both inserted the needle into the patient and also delivered the drug. One disadvantage of this approach is that the force required for drug delivery is typically much greater than the force required for needle insertion. So, the use of a single spring for both drug delivery and needle insertion could result in a level of noise and shock on needle insertion that could significantly frighten the patient and compromise reliability of the autoinjector.

Modern autoinjector designs typically employ two separate springs: an insertion spring to insert the needle and a delivery spring to deliver the drug. This allows both springs to be optimised for their specific purposes. To prevent premature delivery of the drug into the wrong body compartment, these springs are sequenced so that the delivery spring is not released until the needle is inserted far enough into the patient to be in the correct compartment. This means that the drug is not pressurised until the needle is effectively fully inserted into the patient.

It is important that the drug is delivered through the needle into the patient only once the needle has been inserted into the appropriate body compartment, for instance the intramuscular or subcutaneous tissue (as appropriate). This is because delivery of a drug into the wrong body compartment can negatively impact the efficacy of the drug and can cause undesirable side effects.

In some autoinjectors, it may be beneficial to agitate the drug before delivery to the patient. This can reduce the likelihood of drugs, such as suspensions, settling, separating, or causing blockages in the needle and thereby inconsistent delivery. Some drugs can exhibit non-Newtonian viscosity and gelling effects, which can result in highly variable delivery rates or even delivery failures. The application of shear and mixing forces to the drug prior to delivery of the drug to the patient can significantly reduce the negative effects of these occurrences.

One approach for agitating a drug before delivery involves a user shaking the autoinjector before use. The disadvantages of this approach include that the energy available is limited by the capability of the user (who may be ill) to shake the product, and that the energy applied can vary dramatically depending on how individual users interpret the user instructions. In addition, many drug suspensions are opaque even when a large portion of the solid element has settled, so it may be impossible for a user to see whether they have shaken a product enough to re-suspend a settled formulation.

It is an object of the present invention to overcome or reduce one or more of the aforementioned problems.

### SUMMARY OF INVENTION

The invention is defined in the appended independent claims, to which reference should now be made. Optional features of the invention are defined in dependent claims. Aspects, embodiments or examples falling outside the scope of the appended independent claims are included for illustrative or explanatory purposes.

According to a first aspect of the present disclosure, there is provided a drug delivery device. The device may comprise a housing portion. The device may comprise a drug container assembly including a drug container containing a drug. The device may comprise a needle through which the drug is dispensed in use. The needle may be moveable relative to the housing portion, for example from a needle pre-insertion position to a needle insertion position. The device may comprise a plunger rod. Optionally, the plunger rod may be spaced from the drug container assembly prior to movement of the needle from the needle pre-insertion position. The device may comprise a first stored energy source. The device may comprise a needle insertion stored energy source. The device may comprise an activation means. The drug container may comprise an outlet through which the drug is dispensed in use. The outlet may be sealed prior to movement of the needle from the needle pre-insertion position. The device may be configured such that movement of the activation means from a first position to a second position releases the needle insertion stored energy source to move the needle from the needle pre-insertion position to the needle insertion position. The device may be configured such that movement of the activation means from the first position to the second position releases the first stored energy source to move the plunger rod towards and relative to the drug container assembly such that the drug container assembly applies a force to the drug before the needle reaches the needle insertion position. Thus, the device may be configured such that movement of the activation means from the first position to the second position releases the needle insertion stored energy source to move the needle from the needle pre-insertion position to the needle insertion position and releases the first stored energy source to move the plunger rod towards and relative to the drug container assembly such that the drug container assembly applies a force to the drug before the needle reaches the needle insertion position.

Advantageously, the drug container assembly applies a force to the drug before the needle reaches the needle insertion position. Designers of autoinjectors typically seek to minimise any initial shock because it could frighten the patient and compromise reliability of the autoinjector. However, analysis performed by the inventors has surprisingly identified that this force can have the positive effects of mixing and introducing shear forces to the drug. This can cause a settled solid to be re-suspended into the liquid component of a suspension drug and can reduce the viscosity of a shear-thinning drug.

In addition, there is an advantage in the initial impact force on the drug being provided before the start of drug delivery through the needle, particularly where a suspension has settled and formed a solid layer over the exit path for the drug into the needle, which can cause a clogging risk at the start of drug delivery. The drug container may continue to apply force to the drug after the needle reaches the needle insertion position.

However, if the time between the initial impact force and the start of drug delivery is too long, then the suspension component of a drug may start to re-settle, and the viscosity of a shear-thinning drug may increase again as the shear forces in the fluid reduce over time.

Another advantage of the first aspect is that movement of the activation means results in both movement of the needle from the needle pre-insertion position to the needle insertion position, and also in the drug container assembly applying the force to the drug before the needle reaches the needle insertion position. Thus, the device according to the first aspect may require only a single user action (movement of the activation means) to perform needle insertion and to mix the drug at an appropriate stage of operation. This may simplify operation of the device. In addition, this may prevent a problem possible when using some prior art devices where the drug is able to settle, separate, or increase in viscosity after mixing of the drug but prior to needle insertion. This problem may be present, for example, where one manually mixes a drug in a prior art device (for example by shaking or tapping the device), then allows the drug to settle (for example while preparing an injection site of a patient) before performing needle insertion and drug delivery.

As the skilled person would understand from this disclosure, the first stored energy source may be a different stored energy source to the needle insertion stored energy source. The first stored energy source may be one or both of distinct from and separate to the needle insertion stored energy source.

The first stored energy source may be or comprise a first spring. The needle insertion stored energy source may be or comprise a needle insertion spring. The first spring may be a different spring to the needle insertion spring.

As the needle insertion stored energy source moves the needle from the needle pre-insertion position to the needle insertion position, the needle insertion stored energy source may expand along a needle insertion stored energy source axis. As the first stored energy source moves the plunger rod towards and relative to the drug container assembly, the first stored energy source may expand along a first stored energy source axis. The needle insertion stored energy source axis may be different to, and optionally parallel with, the first stored energy source axis.

According to the first aspect, the plunger rod is spaced from the drug container assembly prior to movement of the needle from the needle pre-insertion position. Unless specified otherwise, the term “spaced” is used herein to mean spatially separated. One or more intermediary components may be located between two components spaced from one another. For example, one or more components may be located between the plunger rod and the drug container assembly prior to movement of the needle from the needle pre-insertion position. For example, a compressible component such as a sponge or foam may be located between the plunger rod and the drug container assembly prior to movement of the needle from the needle pre-insertion position. Alternatively, or in addition, an air gap may be located between the plunger rod and the drug container assembly prior to movement of the needle from the needle pre-insertion position. Thus, the plunger rod may be spaced from the drug container assembly prior to movement of the needle from the needle pre-insertion position solely, or partially, by an air gap.

The plunger rod may be spaced from the drug container assembly prior to movement of the needle from the needle pre-insertion position such that, in use, the plunger rod is configured to accelerate substantially unresisted towards the drug container assembly under the action of the first stored energy source. Advantageously, this may allow the allow the drug container assembly to apply a larger force to the drug than if acceleration of the plunger rod towards the drug container assembly were substantially resisted.

The drug delivery device may comprise a housing. The housing may house the drug container assembly. The housing may house the needle in the needle pre-insertion position. The housing may house the plunger rod. The housing may house the first stored energy source. The housing may house the needle insertion stored energy source. The housing may be configured to be held by a user, for example during operation. The drug delivery device may be a hand-held drug delivery device. Advantageously, the housing may make the drug delivery device easier to use. Advantageously, the housing may help protect components housed by the housing, for example from external forces (such as impact forces if the device is dropped) or external contaminants.

As used herein, the term “needle pre-insertion position” refers to a position of the needle prior to movement of the activation means from the first position to the second position. In the needle pre-insertion position, the housing may house the entire needle. The needle pre-insertion position may be distal of the needle insertion position. The needle may be configured to move proximally from the needle pre-insertion position to the needle insertion position.

As used herein, the term “needle insertion position” refers to a position of the needle after movement of the activation means from the first position to the second position. In the needle insertion position, the housing may house a portion, for example a distal portion, of the needle. In the needle insertion position, a portion, for example a proximal portion, may extend out of the housing. The drug delivery device may be configured to dispense the drug only after the needle has left the needle pre-insertion position, for example only after the needle has reached the needle insertion position. The drug delivery device may be configured to dispense the drug only whilst the needle is in the needle insertion position.

The entirety of the force applied by the drug container assembly to the drug may be applied before the needle reaches the needle insertion position. Alternatively, the drug container assembly may start to apply the force to the drug before the needle reaches the needle insertion position but not complete applying the force to the drug until after the needle has reached the needle insertion position. In either case, the drug container assembly applies a force to the drug before the needle reaches the needle insertion position. Unless otherwise stated, references herein to an application of the force from the drug container assembly to the drug may refer to beginning the application of the force from the drug container assembly to the drug or the application of the entire force from the drug container assembly to the drug.

The drug container assembly may apply the force, or start to apply the force, to the drug after the needle has moved from the needle pre-insertion position. Advantageously, this may reduce a time period between applying the force to the drug and the needle reaching the needle insertion position. In addition, by applying the force during the needle insertion process, the likelihood of the drug settling, separating, or substantially increasing in viscosity prior to delivery may be reduced.

Movement of the activation means from the first position to the second position may cause the first stored energy source to move the plunger rod towards the drug container assembly. This movement of the plunger rod towards the drug container assembly may cause the plunger rod or an impacting component to impact an impact surface of the drug container assembly. This impact of the impact surface may cause the drug container assembly to apply force, for example the force, to the drug. Advantageously, causing the plunger rod to impact the impact surface may eliminate the need for the impacting component. Thus, fewer components may be needed, and the drug delivery device may be more compact.

The impact surface may be a surface of the drug container. The plunger rod or the impacting component may be spaced from the impact surface prior to movement of the needle from the needle pre-insertion position. For example, the plunger rod or the impacting component may be spaced from the impact surface by at least 1, 2, 3, 5, 7, or 10 mm prior to movement of the needle from the needle pre-insertion position. Advantageously, a greater separation between the plunger rod or the impacting component and the impact surface may allow the plunger rod or the impacting component to accelerate over a greater distance before impacting the impact surface. This may allow a more forceful impact. A more forceful impact may result in greater mixing of the drug. It may be particularly preferable that the plunger rod or the impacting component is spaced from the impact surface by at least 5 mm prior to movement of the needle from the needle pre-insertion position. This may allow a sufficiently forceful impact.

The plunger rod or impacting component may impact the impact surface with a momentum difference. This momentum difference between the plunger rod or impacting component and the impacting surface may be at least 0.005 or 0.01 kgms⁻¹, and preferably at least 0.02 kgms⁻¹. Advantageously, a greater momentum difference between the plunger rod or impacting surface and the impact surface may allow for a larger energy transfer to the drug. The larger energy transfer to the drug may result in greater mixing of the drug.

The plunger rod or impacting surface may impact the impact surface at a velocity relative to the impact surface of at least 5 ms⁻¹ and preferably at least 10 or 13 ms⁻¹. Advantageously, a larger relative velocity between the plunger rod or impacting surface and the impact surface may allow for a larger transfer of energy to the drug. This may result in greater mixing of the drug.

The drug container assembly may comprise a piston. In use, the piston may be configured to move relative to the drug container. Movement of the piston relative to the drug container may dispense the drug through one or both of the outlet of the drug container and the needle. In use, the drug may be dispensed through the outlet of the drug container and through the needle. In use, movement of the piston relative to the drug container may dispense the drug through the outlet of the drug container and then through the needle. In use, the drug may be dispensed through the outlet of the drug container and the needle simultaneously, for example where the needle extends through the outlet during dispensing of the drug. In use, movement of the piston relative to the drug container may dispense the drug through the outlet of the drug container and the needle simultaneously, for example where the needle extends through the outlet during dispensing of the drug. The piston may comprise, or consist of, a polyethylene material such as HDPE or LDPE, though other materials are possible.

The piston may be coated in a lubricating material. The lubricating material may comprise, or consist of, a silicone material. Advantageously, the lubricating material may reduce the friction between the piston and the drug container while the piston moves relative to the drug container.

A surface of the piston may be configured to act as the impact surface. In use, the plunger rod or impacting component may impact the impact surface of the piston. The piston may then apply the force, or start to apply the force, to the drug. That is, the force applied to the drug by the drug container assembly may be applied by the piston. For example, the plunger rod or impacting component may impact the impact surface of the piston and this may cause the piston to move relative to the drug. This may cause another surface of the piston, for example a surface of the piston substantially opposing the impact surface of the piston, to apply the force, or start to apply the force, to the drug, for example by impacting the drug. Advantageously, it may be convenient for the piston to apply the force, or start to apply the force, to the drug, particularly if the piston is already configured to contact the drug and move relative to the drug. Advantageously, the plunger rod or impacting component impacting an impact surface of the piston may allow a force to be transferred from the first stored energy source to the drug through fewer components. This may lead to less dissipated energy and larger forces acting upon the drug. This may result in greater mixing of the drug.

After the needle has moved from the needle pre-insertion position, for example after the needle has moved from the needle pre-insertion position to the needle insertion position, the first stored energy source may be configured to move the piston relative to the drug container, for example to dispense the drug through the needle. Advantageously, the movement of the piston may not begin until the needle is in the needle insertion position, thus the drug is not delivered through the needle until the needle is at a sufficient minimal length, minimising incorrect anatomical deliveries and premature delivery.

In addition to the outlet, the drug container may comprise an opening. The opening of the drug container may substantially oppose the outlet of the drug container. An opening seal may be provided across the opening of the drug container. The opening seal may close the opening prior to movement of the needle from the needle pre-insertion position. The opening seal may be configured to be ruptured or removed during or prior to use of the drug delivery device. The opening seal may comprise of a sealing foil. Advantageously, the opening seal may act as a microbial barrier and may reduce the ingress of contaminants into the drug container, for example up until it is ruptured or removed.

Prior to movement of the needle from the needle pre-insertion position, the piston may be situated within the drug container, for example between the opening and the drug or between the opening seal and the drug. The piston may contact an internal surface of the drug container. The piston may provide a sealing interface with an internal surface of the drug container so as to prevent the drug from passing from one side of the piston to the other. Locating the piston between the drug and the opening seal may advantageously result in a piston which would not have to form a microbial seal. This may lower the interference requirements of the piston to the drug container. The lower interference requirements may result in lower friction force between the piston and the drug container. This may result in a lower required spring force. The lower required spring force may allow for a smaller spring to be selected, thus decreasing the size of the drug delivery device. Advantageously, the lower interference requirements may reduce the dissipation of energy from drug container assembly to the drug. This may allow for a larger energy transfer to the drug. The larger energy transfer to the drug may result in greater mixing of the drug.

Movement of the first stored energy source may move the plunger rod towards the piston so as to unseal the opening seal, for example by rupturing, piercing, moving or removing the opening seal. The plunger rod may be coupled to a piercing component. The piercing component may pierce the opening seal during movement of the plunger rod towards the piston. Advantageously configuring the plunger rod to be capable of unsealing the opening seal omits the requirement of an additional unsealing element. This reduces the number of components required which may lead to a cheaper, smaller, more compact device.

The drug container may comprise a polymer. The drug container may comprise one or more of a polyethylene polymer and a cyclic olefin polymer. Advantageously, polymer drug containers may be less susceptible to cracking and may be able to withstand higher forces than those of glass. Withstanding higher forces may allow higher forces to be transferred to the drug. This may assist in the mixing of the drug.

Conventionally, it is often desirable to minimise a volume of a gas bubble such as an air bubble, or eliminate the gas bubble completely, within a drug container of a drug delivery device. This is because minimising the volume of the gas bubble may minimise a risk of injecting a significant volume of gas into a patient. In addition, it is often desirable to minimise the volume of the gas bubble where the drug is more susceptible to degradation in the presence of this gas. For example, where the gas comprises oxygen, like air, and the drug is susceptible to oxidation, it may be desirable to minimise the volume of the gas bubble in the drug container. However, contrary to convention in the field of this invention, the inventor has found that the presence of a gas bubble within the drug container may be beneficial. Specifically, the gas bubble may aid mixing of the drug. The gas bubble may aid mixing of the drug when present in a device falling within the scope of the appended independent claims. The gas bubble may have two properties which may significantly aid mixing: a very different specific gravity to the drug, and a level of compressibility that the drug does not have. High speed video and other analytical techniques performed by the inventors have identified rapid compression, expansion, movement, breakup and dispersal of the gas bubble causing turbulent movement throughout the drug and aiding mixing of the drug.

Thus, in addition to the drug within the drug container, a gas bubble may be present within the drug container. As used herein, the term “gas bubble” is used to refer to an undissolved volume of gas. The gas bubble in the drug container may not necessarily be surrounded by the drug. A portion of the gas bubble may be in contact with the drug. A second portion of the gas bubble may be in contact with the drug container. Advantageously, the gas bubble may assist the mixing of the drug. Without wishing to be bound by theory, it is understood that a difference in densities between the gas bubble and the drug may assist in the mixing of the drug. Upon application of the force to the drug by the drug container assembly, the gas bubble may become dispersed within the drug container and be able to travel within the drug container and disturb any settled drug particles. The disturbance of any potentially settled particles may manipulate the drug into a state that is more homogenous than prior to the application of force.

The force applied to the drug by the drug container assembly may compress the gas bubble. Compression of the gas bubble may result in one or both of increasing a pressure of the gas bubble and reducing a volume occupied by the gas bubble. The force applied to the drug by the drug container assembly may compress the gas bubble rapidly. Following compression of the gas bubble, the gas bubble may divide, shatter, or ‘explode’, into multiple secondary gas bubbles. These secondary gas bubbles may travel through the drug and help to mix the drug.

As would be appreciated by one skilled in the art, there is a compromise to be made in the size of the gas bubble present within the drug container. As the volume of the gas bubble is increased, then size of the drug container and the device must also increase to accommodate the gas bubble. In addition, a gas bubble which is too large may, in some embodiments, act as a cushion or damper which actually reduces the force applied to the drug by the drug container assembly. However, a gas bubble which has a small volume may not be able to significantly aid mixing of the drug in the drug container. The compromise which must be made relating to the size of the gas bubble may be affected by the volume of drug in the drug container, the pressure of the gas bubble prior to compression, and the shape of the drug container. Prior to movement of the needle from the needle pre-insertion position, the gas bubble may occupy a volume of at least 50 or 100 mm³ at room temperature and atmospheric pressure. Prior to movement of the needle from the needle pre-insertion position, the gas bubble may occupy a volume of no more than 5000 or 2000 mm³ at room temperature and atmospheric pressure. Prior to movement of the needle from the needle pre-insertion position, the gas bubble may occupy a volume of between 50 and 5000 mm³, preferably between 100 and 2000 mm³, at room temperature and atmospheric pressure.

Prior to movement of the needle from the needle pre-insertion position, the gas bubble may occupy at least 5% of the volume of the drug container. Prior to movement of the needle from the needle pre-insertion position, the gas bubble may occupy no more than 30% of the volume of the drug container.

The outlet of the drug container may be sealed prior to movement of the needle from the needle pre-insertion position. The outlet may be sealed by an outlet seal. The device may comprise an unsealing mechanism for unsealing the outlet of the drug container in use. The unsealing mechanism may be configured to engage with the outlet seal so as to unseal the outlet. For example, the unsealing mechanism may be configured to rupture, pierce, move, or remove the outlet seal. The unsealing mechanism may be configured to unseal the outlet after the needle has moved from the needle pre-insertion position. Advantageously, this ensures that the outlet is unable to be unsealed prematurely, for example while the drug delivery device is not being used, or such that the drug is delivered in the wrong body compartment.

The unsealing mechanism may unseal the outlet after the drug container assembly applies the force to the drug. Advantageously, this may prevent prematurely dispensing the drug due to the application of the force to the drug. For example, the unsealing mechanism may be configured to unseal the outlet at least 5 ms and preferably at least 12 ms after the force is applied or after the start of the application of the force. Unsealing the outlet at least a predetermined time after the force has been applied may advantageously ensure that there has been sufficient time for the mixing to occur prior to drug delivery.

The unsealing mechanism may unseal the outlet after the needle has travelled at least a predetermined distance from the needle pre-insertion position, for example at least 10 or 20 mm from the needle pre-insertion position, or at least 50%, 75% or 90% of a distance from the needle pre-insertion position to the needle insertion position. Advantageously, this may ensure that there has been sufficient time for the drug to mix before the outlet is unsealed and the drug is dispensed.

The unsealing mechanism may be operated to unseal the outlet after the needle has reached the needle insertion position. Advantageously, this may reduce a risk of the drug being dispensed through the needle prematurely, causing drug delivery into the wrong body compartment.

The unsealing mechanism may be configured to unseal the outlet at least 10 or 20 ms after the activation means has reached the second position. Advantageously, this may allow for sufficient time to elapse to ensure the drug has been sufficiently mixed, before delivering the drug through the outlet.

The unsealing mechanism may comprise the needle. In use, the needle, for example a distal end of the needle, may pierce the outlet seal to unseal the outlet. At least a portion of the outlet seal, and optionally the drug container, may move relative to the needle to cause the needle to pierce the outlet seal. At least a portion of the outlet seal, and optionally the drug container, may move proximally relative to the needle to cause the distal end of the needle to pierce the outlet seal. At least a portion of the outlet seal, and optionally the drug container, may move relative to the needle after the needle has moved from the needle pre-insertion position, or after the needle has reached the needle insertion position, to cause the needle to pierce the outlet seal. This may cause the needle to pierce the outlet seal and unseal the outlet after the needle has left the needle pre-insertion position, or after the needle has reached the needle insertion position. In one example, after the needle has reached the needle insertion position, the drug container and outlet seal move proximally relative to the needle to cause the distal end of the needle to pierce the outlet seal and unseal the outlet.

In use, the outlet seal may flex. For example, the outlet seal may flex outwardly or away from the drug container. The outlet seal may flex under an increase in pressure in the drug container, for example as the piston moves relative to the drug container. This flexing may move the outlet seal, or at least a portion of the outlet seal, relative to the needle to cause the needle to pierce the outlet seal.

The outlet may initially be sealed by a valve. In use, the valve may be opened so as to unseal the outlet. The valve may be opened as a consequence, for example an indirect consequence, of moving the activation means from the first position to the second position.

The drug container assembly may apply or start to apply the force to the drug at least 5 or 10 ms before the needle reaches the needle insertion position. Advantageously, this may allow for a greater time for the force to mix the drug prior to delivery of the drug through the outlet.

The drug container assembly may apply or start to apply the force to the drug before the needle has travelled a predetermined distance from the needle pre-insertion position, for example before the needle has travelled a distance of 5 or 10 mm from the needle pre-insertion position, or before the needle has travelled 25% or 50% of a distance from the needle pre-insertion position to the needle insertion position. Advantageously, this may allow for a greater time for the force to mix the drug prior to delivery of the drug through the outlet.

The first stored energy source may be released to move the plunger rod towards and relative to the drug container assembly. The first stored energy source may be released after movement of the needle from the needle pre-insertion position. The first stored energy source may be released before the needle reaches the needle insertion position. Advantageously, this may ensure that the force applied by the drug container assembly to the drug is applied at an appropriate stage of operation of the device, for example after the needle has left the needle pre-insertion position but before the needle reaches the needle insertion position.

The drug delivery device may comprise a housing. The activation means may comprise a skin sensor element that is moveable relative to the housing. Movement of the activation means from the first position to the second position may comprise a distal movement of the skin sensor element relative to the housing. The housing may be configured to be held by the user.

When the needle is in the needle pre-insertion position, the skin sensor element may be biased into a proximal position relative to the housing, for example by a skin sensor element biasing spring.

The activation means may be configured such that pressing the skin sensor element against the injection site results in the distal movement of the skin sensor element relative to the housing. The activation means comprising the skin sensor element may advantageously simplify operation of the device. The user may simply be able to operate the device by pressing the skin sensor against the injection site, thereby moving the activation means from the first position to the second position.

The drug may comprise a non-Newtonian fluid. The drug may comprise a non-Newtonian gel. The drug may exhibit shear-thinning properties. For example, the drug may exhibit time-dependent shear-thinning properties. The drug may comprise a thixotropic fluid. The drug may comprise a thixotropic gel.

The drug may comprise a solute dispersed in a solvent, for example salt dispersed in water.

In the lowest energy state of the drug in the drug container, the drug may not be homogeneous. For example, the drug may not be homogeneous in the lowest energy state of the drug during storage. As used here, the term “during storage” describes the conditions in which the drug is intended to be transported and stored before use. Thus, the term “during storage” may refer to one or both of room temperature and atmospheric pressure. Alternatively, if the drug is refrigerated prior to use, then the term “during storage” may refer to a temperature lower than room temperature.

If left undisturbed for a sufficient period of time, for example greater than 24 hours, the drug may naturally resort to a non-homogeneous state. If left undisturbed for a sufficient period of time, for example greater than 24 hours, the drug may stratify, or separate into layers. A concentration of a component of the drug may vary between these layers. As an example, where the drug comprises a suspension, if left undisturbed for a sufficient period of time, a lower portion of the suspension may have a greater concentration of the solid particles than an upper portion of the drug. This may occur naturally under the action of gravity. In this context, the terms “lower portion” and “upper portion” may be defined relative to the direction in which gravity acts rather than fixed, designated portions of the drug container. Thus, where gravity acts in a direction from the upper portion to the lower portion of the drug container, gravity may act to increase the concentration of the suspension in the lower portion of the drug container compared with the upper portion.

Advantageously, the device may be particularly suitable for drugs having one or more of the features described above. This may be because such drugs may benefit most from being mixed prior to delivery.

Upon application of the force to the drug by the drug container assembly, the dynamic viscosity of the drug may decrease. All references herein to dynamic viscosities refer to dynamic viscosities measured using the Hagen-Poiseuille equation, though any suitable process could be used. For example, after the drug container assembly applies the force to the drug, the dynamic viscosity of the drug may reduce to less than 10 cP and more preferably to less than 8 or 4 cP at room temperature. The dynamic viscosity of the drug may reduce from greater than 15, 20 or 50 cP to less than 10, 8 or 4 cP at room temperature after the force has been applied to the drug. The reduction in dynamic viscosity may advantageously decrease the force required to deliver the drug through the needle. The decrease in dynamic viscosity may allow the drug to pass through a smaller gauge needle, the smaller gauge needle potentially being less noticeable to a patient during needle insertion. The reduction in dynamic viscosity may also allow a predetermined volume of the drug to be delivered in a smaller time period.

The drug delivery device may be an autoinjector. The device may automate needle insertion and drug delivery. Advantageously, the device being an autoinjector may simplify use of the device and may allow a patient to use the device themselves.

According to a second aspect of the present disclosure, there is provided a method of mixing a drug. The drug may be mixed in a drug delivery device. The device may comprise a drug dispensing mechanism. The drug dispensing mechanism may include a stored energy source. The device may comprise a drug container containing a drug. The method may comprise applying a force to the drug using the stored energy source to mix the drug in the drug container and/or increase a homogeneity of the drug in the drug container and/or to reduce a dynamic viscosity of the drug in the drug container.

Advantageously, mixing, increasing the homogeneity of, or reducing a dynamic viscosity of, the drug may reduce the likelihood of effects mentioned before such as clogging of a needle or outlet of a drug delivery device and inconsistent pharmacokinetic profiles.

Features described in relation to the drug delivery device above, such as the drug delivery device of the first aspect, may be applicable to the method or the drug delivery device of the second aspect.

The drug delivery device may be the drug delivery device according to the first aspect. In this case, the stored energy source of the second aspect may be, or may comprise, the first stored energy source of the first aspect.

Applying the force to the drug may comprise impacting a plunger rod or an impacting component against an impact surface. As explained above with reference to the first aspect, the drug delivery device may comprise the plunger rod or the impacting component and the impact surface. Configuring the plunger rod to impact the impacting surface may advantageously reduce the number of components and reduce the size of the drug delivery device.

The plunger rod or impacting component may be spaced from the impact surface prior to applying the force to the drug. For example, the plunger rod may be held at a distance of at least 1, 2, 3, 5, 7 or 10 mm from the impact surface prior to applying the force to the drug. Advantageously, the separation of the plunger rod or impacting component and impact surface may allow for greater acceleration of the plunger rod or impacting component relative to the impact surface to enhance the momentum difference upon impact. This may increase mixing of the drug due to the increased impact energy.

The drug delivery device may comprise an activation means. The method of mixing the drug may comprise moving the activation means from a first position to a second position to release the stored energy source to apply the force to the drug.

An outlet may be present in the drug container through which the drug is dispensed in use. Initially the outlet may be sealed. The method may comprise unsealing the outlet. The outlet may be unsealed after applying the force to the drug. Configuring the outlet to be sealed before and during the force being applied may allow for the force to be applied to the drug without the drug being dispensed from the drug container.

The outlet may be unsealed automatically after moving the activation means from the first position to the second position. Advantageously, this may mean that no further actions are required by the user following moving the activation means from the first position to the second position in order to unseal the outlet.

The drug delivery device may comprise a needle. The method of mixing the drug may comprise moving the needle from a first needle location to a second needle location. The first needle location may be configured such that the needle resides within the drug delivery device. The second needle location may be positioned such that the needle protrudes from the drug delivery device. The first needle location may correspond to, or be, the needle pre-insertion position of the needle of the device of the first aspect. The second needle location may correspond to, or be, the needle insertion position of the needle of the device of the first aspect. Advantageously, moving the needle from a first location to a second location allows for a more user-friendly needle insertion mechanism, leading to better patient compliance than a manual needle insertion mechanism.

Movement of the needle from the first location to the second location may be caused by movement of the activation means from the first position to the second position. Advantageously, this may mean that no further actions are required by the user following moving the activation means from the first position to the second position in order to move the needle from the first needle location to the second needle location.

A needle insertion stored energy source, such as the needle insertion stored energy source described in relation to the first aspect, may move the needle from the first location to the second location.

As described in relation to the device of the first aspect, the device may comprise a housing and the activation means may comprise a skin sensor element movable relative to the housing. Movement of the activation means from the first position to the second position may comprise a distal movement of the skin sensor element relative to the housing. The method may comprise pressing the skin sensor element against an injection site. This may result in the distal movement of the skin sensor element relative to the housing. Prior to movement of the needle from the first needle location, or the needle pre-insertion position, the skin sensor element may be biased into a proximal position relative to the housing.

The method of mixing the drug may comprise applying the force to the drug before the needle reaches the second needle location. This may advantageously allow a greater time period for mixing of the drug following application of the force to the drug.

The force may be applied after the needle has moved from the first needle location. This may advantageously ensure that the mixing of the drug occurs in a timely manner relative to the delivery of the drug. This may ensure that the mixing effects on the drug are not undone or reversed before delivery.

As used herein, the term “injection site” may refer to the area of a patient through which a drug is to be delivered.

As used herein, the term “room temperature” refers to a temperature of around 20 degrees Celsius.

As used herein, front and proximal are used to refer to the same end of the device. The front end may be placed on the injection site during use. Similarly, rear and distal are used to refer to the same end of the device. The rear end of the device may be opposite to the front end of the device. The rear end may be furthest from the injection site during use.

The invention is defined by the claims. However, below is a non-exhaustive list of numbered, non-limiting clauses. Any one or more of the features of these clauses may be combined with any one or more features of an example, embodiment, or aspect described herein, such as any one or more features of the first and second aspects described above.
1. A drug delivery device comprising:
a housing portion; a drug container assembly including a drug container containing a drug; a needle through which the drug is dispensed in use, the needle being moveable relative to the housing portion from a needle pre-insertion position to a needle insertion position; a plunger rod spaced from the drug container assembly prior to movement of the needle from the needle pre-insertion position; a first stored energy source; a needle insertion stored energy source; and an activation means,
wherein the drug container comprises an outlet through which the drug is dispensed in use, the outlet being sealed prior to movement of the needle from the needle pre-insertion position,
and wherein the device is configured such that movement of the activation means from a first position to a second position releases the needle insertion stored energy source to move the needle from the needle pre-insertion position to the needle insertion position and releases the first stored energy source to move the plunger rod towards and relative to the drug container assembly such that the drug container assembly applies a force to the drug before the needle reaches the needle insertion position.
2. A drug delivery device according to clause 1, wherein the drug container assembly applies the force to the drug after the needle has moved from the needle pre-insertion position.
3. A drug delivery device according to clause 1 or 2, wherein movement of the activation means from the first position to the second position causes the first stored energy source to move the plunger rod towards the drug container assembly such that the plunger rod or an impacting component impacts an impact surface of the drug container assembly such that the drug container assembly applies the force to the drug.
4. A drug delivery device according to clause 3, wherein the plunger rod or the impacting component is spaced from the impact surface by at least 1, 2, 3, 5, 7 or 10 mm prior to movement of the needle from the needle pre-insertion position.
5. A drug delivery device according to any of clauses 3 to 4, wherein, following movement of the activation means from the first position to the second position, the plunger rod or the impacting component impacts the impact surface with a momentum difference between the plunger rod or the impacting component and the impact surface of at least 0.005, 0.01, or 0.02 kgms⁻¹.
6. A drug delivery device according to any of clauses 3 to 5, wherein, following movement of the activation means from the first position to the second position, the plunger rod or the impacting component impacts the impact surface with a velocity relative to the impact surface of at least 5, 10 or 13 ms⁻¹.
7. A drug delivery device according to any preceding clause, wherein the drug container assembly comprises a piston and, in use, the piston moves relative to the drug container to dispense the drug through the needle.
8. A drug delivery device according to any of clauses 3 to 6, wherein the drug container assembly comprises a piston, the impact surface is a surface of the piston, and, in use, the piston moves relative to the drug container to dispense the drug through the needle.
9. A drug delivery device according to clause 7 or 8, wherein, after movement of the needle from the needle pre-insertion position to the needle insertion position, the first stored energy source is configured to move the piston relative to the drug container to dispense the drug through the needle.
10. A drug delivery device according to any preceding clause, wherein the drug container comprises, in addition to the outlet, an opening sealed by an opening seal prior to movement of the needle from the needle pre-insertion position.
11. A drug delivery device according to any of clauses 7 to 9, wherein the drug container comprises, in addition to the outlet, an opening sealed by an opening seal prior to movement of the needle from the needle pre-insertion position and wherein, prior to movement of the needle from the needle pre-insertion position, the piston is located within the drug container and between the drug and the opening seal.
12. A drug delivery device according to clause 11, wherein movement of the activation means from the first position to the second position causes the first stored energy source to move the plunger rod towards the piston so as to pierce the opening seal.
13. A drug delivery device according to any preceding clause, wherein the drug container comprises a polymer.
14. A drug delivery device according to clause 13, wherein the drug container comprises one or more of a polypropylene polymer and a cyclic olefin polymer.
15. A drug delivery device according to any preceding clause, wherein the drug container contains a gas bubble in addition to the drug.
16. A drug delivery device according to clause 15, wherein, prior to movement of the activation means from the first position to the second position, the gas bubble has a volume of at least 120 mm³ at room temperature.
17. A drug delivery device according to any preceding clause, wherein the device comprises an unsealing mechanism for unsealing the outlet.
18. A drug delivery device according to clause 17, wherein the unsealing mechanism is configured to unseal the outlet after the needle has moved from the needle pre-insertion position.
19. A drug delivery device according to clause 17 or 18, wherein the unsealing mechanism is configured to unseal the outlet after the drug container assembly applies the force to the drug.
20. A drug delivery device according to any of clauses 17 to 19, wherein the unsealing mechanism is configured to unseal the outlet after the needle has travelled at least 50%, 75% or 90% of a distance from the needle pre-insertion position to the needle insertion position, for example wherein the unsealing mechanism is configured to unseal the outlet after the needle has reached the needle insertion position.
21. A drug delivery device according to any of clauses 17 to 20, wherein the unsealing mechanism is configured to unseal the outlet at least 5, 10 or 12 ms after the drug container assembly applies the force to the drug.
22. A drug delivery device according to any of clauses 17 to 21, wherein the unsealing mechanism is configured to unseal the outlet at least 10 or 20 ms after movement of the activation means from the first position to the second position.
23. A drug delivery device according to any preceding clause, wherein the drug container assembly applies the force to the drug at least 5, 10 or 12 ms before the needle reaches the needle insertion position.
24. A drug delivery device according to any preceding clause, wherein the drug container assembly applies the force to the drug before the needle has travelled 25% or 50% of a distance from the needle pre-insertion position to the needle insertion position.
25. A drug delivery device according to any preceding clause, wherein the first stored energy source is released to move the plunger rod towards and relative to the drug container assembly after movement of the needle from the needle pre-insertion position but before the needle reaches the needle insertion position.
26. A drug delivery device according to any preceding clause, wherein the device comprises a housing and the activation means comprises a skin sensor element movable relative to the housing, and wherein movement of the activation means from the first position to the second position comprises a distal movement of the skin sensor element relative to the housing.
27. A drug delivery device according to clause 26, wherein, prior to movement of the needle from the needle pre-insertion position, the skin sensor element is biased into a proximal position relative to the housing.
28. A drug delivery device according to clause 26 or 27, wherein the activation means is configured such that pressing the skin sensor element against an injection site results in the distal movement of the skin sensor element relative to the housing.
29. A drug delivery device according to any preceding clause, wherein the drug comprises a non-Newtonian fluid or gel.
30. A drug delivery device according to any preceding clause, wherein the drug comprises a shear-thinning fluid or gel.
31. A drug delivery device according to any preceding clause, wherein the drug comprises a thixotropic fluid or gel.
32. A drug delivery device according to any preceding clause, wherein the drug comprises a solute dispersed in a solvent.
33. A drug delivery device according to any preceding clause, wherein the drug is not homogeneous in its lowest energy state.
34. A drug delivery device according to any preceding clause, wherein the force applied to the drug reduces a dynamic viscosity of the drug.
35. A drug delivery device according to clause 34, wherein the force applied to the drug reduces the dynamic viscosity of the drug to less than 10 cP and more preferably to less than 8 or 4 cP at room temperature.
36. A drug delivery device according to clause 35, wherein the force applied to the drug reduces the dynamic viscosity of the drug from greater than 10 cP to less than 8 or 4 cP at room temperature.
37. A drug delivery device according to any preceding clause, wherein the device is an autoinjector.
38. A method of mixing a drug in a drug delivery device, the device comprising:
   a drug dispensing mechanism including a stored energy source, and a drug container containing a drug, and the method comprising:
   applying a force to the drug using the stored energy source to mix and increase a homogeneity of the drug in the drug container and/or to reduce a dynamic viscosity of the drug in the drug container.
39. A method according to clause 38, wherein applying the force to the drug comprises impacting a plunger rod or an impacting component against an impact surface.
40. A method according to clause 39, wherein, prior to applying the force to the drug, the plunger rod or the impacting component is spaced from the impact surface by at least 1, 2, 3, 5, 7, or 10 mm.
41. A method according to any one of clauses 38 to 40, wherein the device comprises an activation means and the method comprises moving the activation means from a first position to second position to release the stored energy source to apply the force to the drug.
42. A method according to clause 41, wherein the drug container comprises an outlet through which the drug is dispensed in use, the outlet being sealed initially, and the method comprises unsealing the outlet.
43. A method according to clause 42, wherein unsealing the outlet occurs after applying the force to the drug.
44. A method according to clause 42 or 43, wherein the outlet is unsealed automatically after moving the activation means from the first position to the second position.
45. A method according to any of clauses 38 to 44, wherein the device comprises a needle and the method comprises moving the needle from a first needle location to a second needle location.
46. A method according to any of clauses 41 to 44, wherein the device comprises a needle and the method comprises moving the needle from a first needle location to a second needle location, and wherein movement of the activation means from the first position to the second position causes movement of the needle from the first needle location to the second needle location.
47. A method according to clause 45 or 46, wherein applying the force to the drug occurs before the needle reaches the second needle location.
48. A method according to clause 45, 46 or 47, wherein applying the force to the drug occurs after the needle has moved from the first needle location.
49. A method according to any of clauses 38 to 48, wherein the method is a method of mixing a drug in a drug delivery device according to any of clauses 1 to 37.

### BRIEF DESCRIPTION OF DRAWINGS

FIG. 1 is a cross-section of a drug delivery device, with the cap covering the proximal end of the device.

FIG. 2 is a cross-section of the drug delivery device of FIG. 1 in the pre-activation position, with the cap and needle shield removed.

FIG. 3 is a cross-section of the drug delivery device of FIG. 1 with the activation means in the distal position relative to the housing and front chassis.

FIG. 4a is an alternative cross-section of the distal end of the drug delivery device of FIG. 1 in the pre-activation position.

FIG. 4b is an alternative cross-section of the distal end of the drug delivery device of FIG. 1 during operation, whereby the drug container holder has been proximally displaced relative to the release pin.

FIG. 4c is an alternative cross-section of the distal end of the drug delivery device of FIG. 1 during operation, whereby the plunger rod has uncoupled from the release pin.

FIG. 4d is an alternative cross-section of the distal end of the drug delivery device during operation, whereby the plunger rod has been proximally displaced relative to the drug container holder.

FIG. 5 is a cross-section view of the drug delivery device of FIG. 1 during operation, whereby the needle has moved proximally from the needle pre-insertion position and the plunger rod has uncoupled from the release pin but remains at a distance from the drug container opening/sealing foil.

FIG. 6 is a cross-section view of the drug delivery device of FIG. 1 whereby the plunger rod has contacted the impacting surface.

FIG. 7 is a cross-section view of the drug delivery device of FIG. 1 whereby the needle has reached the needle insertion position.

FIG. 8 is a cross-section view of the drug delivery device of FIG. 1 following unsealing of the outlet of the drug container.

FIG. 9 is a cross-section view of the drug delivery device of FIG. 1 upon completion of drug delivery through the needle.

### DETAILED DESCRIPTION

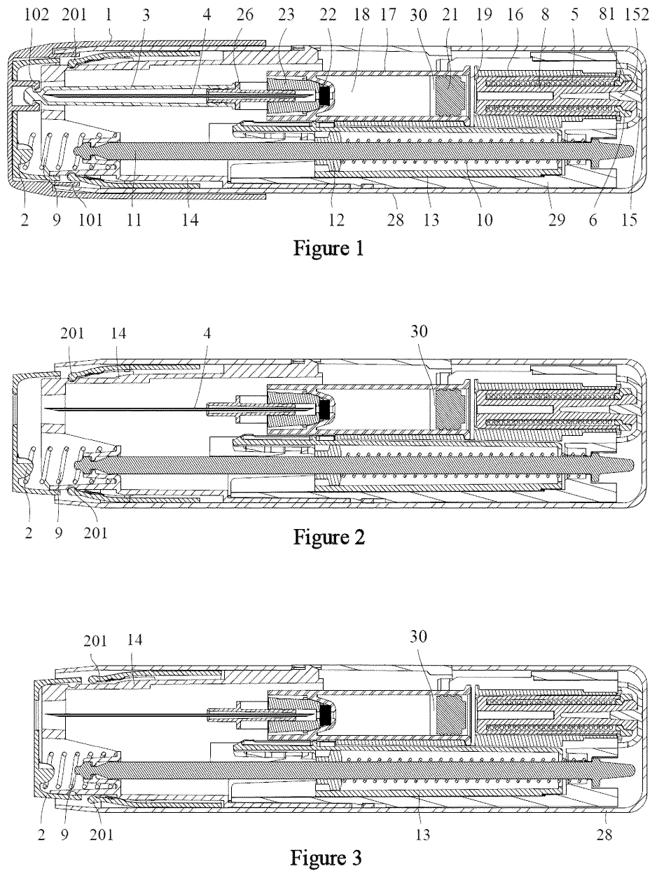

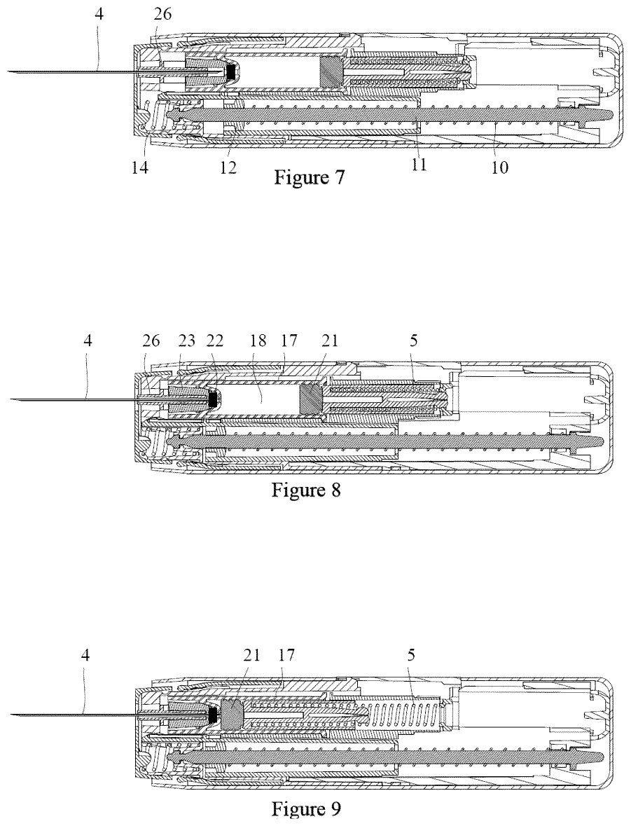

FIG. 1 is a cross-section of a drug delivery device in an initial, pre-activation position, prior to use. The device shown in FIG. 1 is a drug delivery device comprising a drug container assembly including a drug container 17 containing a drug 18 and an air bubble 30. The drug delivery device is an autoinjector and comprises a housing 28 that houses the drug container assembly. The drug delivery device further comprises a needle 4 through which the drug 18 is dispensed in use, the needle 4 being moveable relative to the housing 28 of the device from a needle pre-insertion position (as shown in FIG. 1) to a needle insertion position (as shown in FIG. 7); a plunger rod 8 positioned spaced from the drug container assembly prior to movement of the needle 4 from the needle pre-insertion position; a first stored energy source 5, which is a helical spring; a needle insertion stored energy source 10, which is another helical spring; and an activation means including a skin contact element, herein referred to as skin sensor element 2.

The drug container 17 comprises an outlet through which the drug 18 is dispensed in use, the outlet being sealed by a sealing element 22 prior to movement of the needle 4 from the needle pre-insertion position.

The device is configured such that movement of the activation means from a first position to a second position releases the needle insertion stored energy source 10 to move the needle 4 from the needle pre-insertion position to the needle insertion position and releases the first stored energy source 5 to move the plunger rod 8 towards and relative to the drug container assembly to apply a force on the drug container 17 such that the drug container assembly applies a force to the drug 18 before the needle 4 reaches the needle insertion position.

The drug container 17 has a generally tubular shape, creating an internal space containing the drug 18. The outlet is provided at a proximal end and a distal opening is provided at a distal end.

In this embodiment, the drug is a non-Newtonian fluid. Specifically, the drug is a thixotropic fluid which tends to increase in viscosity if left undisturbed for a sufficiently long time. This could make dispensing the drug difficult and can result in the drug clogging the needle. The drug also tends to separate into layers if left undisturbed for a sufficiently long period of time, where a concentration of a component of the drug varies between the layers. Applying the force to the drug helps to reduce the viscosity and mix the drug to increase its homogeneity before delivering the drug to the patient.

A piston 21 is positioned between the drug 18 and the distal opening. In this embodiment, the drug container 17 is formed from a cyclic olefin polymer. The piston 21 comprises an impacting surface on its distal face. During use, the piston 21 creates a seal with the interior wall of the drug container 17. This prevents the drug 18 from passing from one side of the piston 21 to the other. The piston 21 is moveable in a proximal direction relative to the drug continuer 17 to deliver the drug 18 through the outlet of the drug container 17, and through the needle 4.

An opening seal 19, such as a sealing foil, seals the distal opening of the drug container 17 in the pre-activation position. The opening seal 19 is arranged to be pierced by the plunger rod 8 in use.

A fluid-tight sealing element, in the form of a rubber plug 22, is provided between the drug container 17 and a sealing retainer 23. The sealing retainer 23 is coupled to the drug container 17 and the rubber plug 22 is coupled to the proximal opening of the drug container 17. In use, the rubber plug 22 is configured to be pierced by the needle 4. The needle 4 is fixed to a needle holder 26 which is slidably coupled to the proximal end of the drug container 17. During use, the drug container assembly is moved proximally relative to the needle holder 26 such that the distal end of the needle 4 pierces through the rubber plug 22 and creates a fluid path for the drug 18 through the needle 4.

The drug container 17 is coupled to a drug container holder 16. The drug container holder 16 houses the plunger rod 8 which comprises a proximal face which is configured to pierce the sealing foil 19 and abut the piston 21. In the pre-activation position, the plunger rod sits around 5 mm from the sealing foil 19. At a distal end, the plunger rod 8 comprises two opposing distal plunger rod arms 81. The first stored energy source 5 is configured to sit around the plunger rod 8 and be located at a proximal end on a face of the plunger rod 8. The distal end of the first stored energy source 5 is located against a spring retainer 6. The spring retainer 6 is coupled to the drug container holder 16 and comprises a central opening large enough for the distal plunger rod arms 81 to travel within.

In the pre-activation position, as shown in FIG. 1, the distal plunger rod arms 81 are configured to reside through the central opening of the spring retainer 6, such that the first stored energy source 5 is in a first compressed state and the proximal face of the plunger rod 8 is separated from the drug container 17 and sealing foil 19.

A release pin 15 is located at the distal end of the device and is coupled to a rear chassis 29. The rear chassis 29 is fixed to the housing. The release pin 15 comprises a proximally extending protrusion 152, which in a first stored position is configured to reside between the two opposing distal plunger rod arms 81.

The needle insertion stored energy source 10 is arranged along a spring shaft 11. The needle insertion stored energy source 10 in this embodiment is a spring so may be referred to as the needle insertion spring 10. The needle insertion spring 10 is located at a distal end against a proximal surface of the rear chassis 29 and at a proximal end against a spring boss 12. The spring boss 12 is configured to be slidably arranged relative to the spring shaft 11. The spring boss 12 is coupled to a spring holder 13 which houses the needle insertion spring 10 and is slidably arranged relative to the spring shaft 11. The spring holder 13 is coupled to the drug container holder 16 such that proximal movement of the spring holder 13 under the biasing force of the needle insertion spring 10 equates into proximal displacement of the drug container assembly. A locking member of the spring holder 13 (not pictured) prohibits the movement of the spring holder 13 relative to the rear chassis 29. The locking member of the spring holder 13 comprises a locking member latch (not pictured) which in the first position is in communication with the activation means.

The activation means comprises a skin sensor 2 at the proximal end of the drug delivery device. The skin sensor 2 is slidably arranged relative to the housing 28 and a front chassis 14, the front chassis 14 being fixed to the housing 28. The skin sensor 2 is biased in a first proximal position (seen in FIG. 1) by a skin sensor spring 9 relative to the housing 28. Where the skin sensor 2 is in the first proximal position a locking member latch of the spring holder 13 is coupled to the front chassis 14.

The sequence of operation of the device shown in FIG. 1 will now be described. The same reference numerals are used to label the same features in all of the Figures.

In the pre-activation position shown in FIG. 1, the cap 1 covers the proximal end of the drug delivery device. The cap 1 is slidably removable from the housing 28 of the device. The cap 1 comprises distally extending protrusions 101 which reside within the housing 28 in the pre-activation position. The cap 1 also comprises removal arms 102 which are arranged to sit around a proximal feature of a needle shield 3. The needle 4 is arranged to sit within the needle shield 3 when the cap 1 is on the device, whereby the needle shield 3 provides a closed sterile environment.

The skin sensor 2 comprises flexible members 201 which reside in a recess on the front chassis 14 when the skin sensor 2 is in the extended position. With the cap 1 covering the proximal end of the device, the extending protrusions 101 prohibit flexible members 201 of the skin sensor 2 from moving from their first position within the front chassis 14, therefore prohibiting premature movement of the skin sensor 2 and premature activation of the device. In a first stage, a user removes the cap 1 from the housing 28 of the drug delivery device, together with the needle shield 3. The device with the cap removed is shown in FIG. 2. The removal of the cap 1 from the drug delivery device removes the distal protrusions 101 from within the housing 28 therefore permitting the flexible members 201 of the skin sensor 2 to disengage from the front chassis 14. The proximal end of the needle 4 is exposed. The skin sensor 2 is biased in the first proximal position by the skin sensor spring 9.

The user operates the drug delivery device by placing the proximal end of the device onto an injection site while holding the housing 28. As the user pushes the housing 28 towards the injection site, the skin sensor 2 is slidably displaced distally within and relative to the housing 28 as shown in FIG. 3. As the skin sensor 2 is displaced distally, the resilient arms 201 of the skin sensor 2 are encouraged to move from the recess on the front chassis 14 and flex into their second position. The skin sensor spring 9 becomes compressed between the skin sensor 2 and front chassis 14.

As the skin sensor 2 is displaced further relative to the housing 28 into a retracted distal position, the locking member latch of the spring holder 13 is released from its first locked position into an aperture of the skin sensor 2. The latch is disengaged from its locking position, into a second unlocked position which permits movement of the spring holder 13 from its first position.

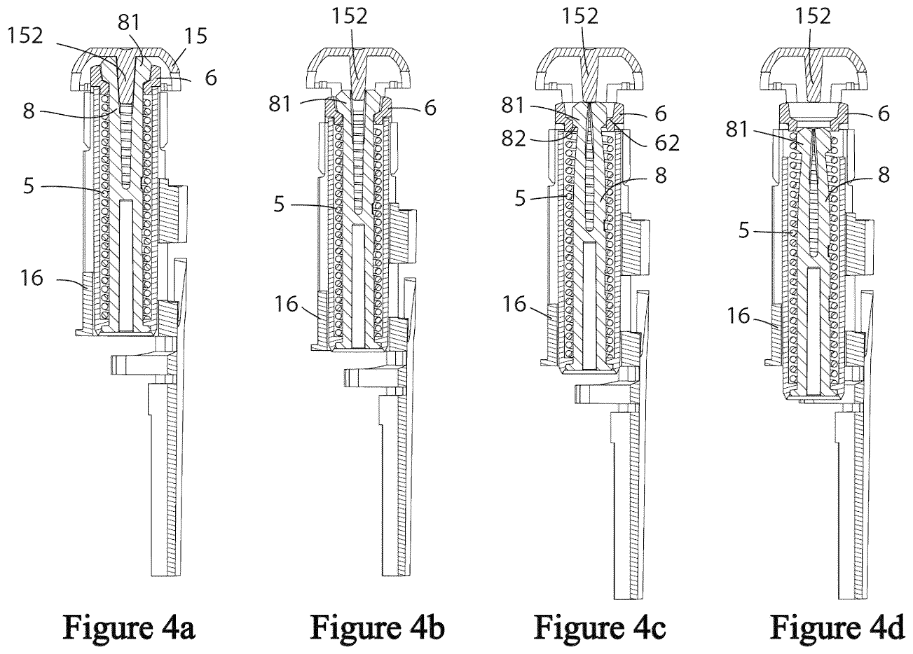

FIGS. 4a-4d are cross-sections of the distal end of the device which show how, in this embodiment, a time delay is provided between activation of the needle insertion spring 10 to move the needle 4 from the needle pre-insertion position and the release of the plunger rod 8 to contact the drug container assembly.

FIG. 4a shows the distal end of the drug delivery device of FIG. 1 in a pre-activation position. In the pre-activation position of the device, as mentioned above, the first stored energy source 5 is held between the plunger rod 8 and spring retainer 6. The biasing force of the first stored energy source 5 encourages proximal movement of the plunger rod 8 relative to the drug container holder 16. This biasing force encourages the two distal plunger rod arms 81 to flex inwardly but they are prevented from flexing inwardly by the protrusion 152 of the release pin 15 located between the two distal plunger rod arms 81. The drug container holder 16 and coupled spring retainer 6 are held in position relative to the rear chassis 29 by the locking member latch of the spring holder 13. The release pin 15 is coupled to the rear chassis 29, therefore prohibiting relative movement between the spring retainer 6 and release pin 15 in the pre-activation position.

The cross-section of FIG. 4b shows the device after movement of the skin sensor 2 of the activation means from the first position to the second position. This movement of the skin sensor 2 results in the locking member latch of the spring holder 13 being released from its first locked position into an aperture of the skin sensor 2 as previously mentioned. This disengages the latch from its locking position into a second unlocked position which permits movement of the spring holder 13 from its first position. The needle insertion spring 10 biases the spring holder 13 proximally relative to the housing 28. Since the spring holder 13 is coupled to the drug container holder 16, the needle insertion spring 10 also moves the drug container assembly proximally relative to the housing 29 and relative to the release pin protrusion 152. The drug container holder 16 therefore slides within the rear chassis 29. The spring retainer 6 and plunger rod arms 81 are also displaced relative to the release pin protrusion 152. The first stored energy source 5 remains compressed.

FIGS. 4c and 4d are further cross-sections showing views of the plunger rod 8 and relative movement between the release pin protrusion 152 and the drug container holder 16. Once the drug container holder 16 has travelled a sufficient distance in the proximal direction, the distal plunger rod arms 81 are disengaged from the release pin protrusion 152 and so are permitted to flex inwards under the biasing force of the first stored energy source 5. This inward flexing is shown in FIG. 4c. Chamfered edges 82 upon the distal plunger rod arms 81 are configured to aid in the inward flexing of the distal plunger rod arms 81 to assist in the plunger rod 8 moving proximally through the inner bore of the spring retainer 6. An opposing chamfered ledge 62 is found on the spring retainer 6 to further encourage the inward flexing of the distal plunger rod arms 81. This inward flexing allows the distal plunger rod arms 81 to travel through the internal bore of the spring retainer 6 and relative to the drug container holder 16 under the action of the first stored energy source 5, as is shown in FIG. 4d.

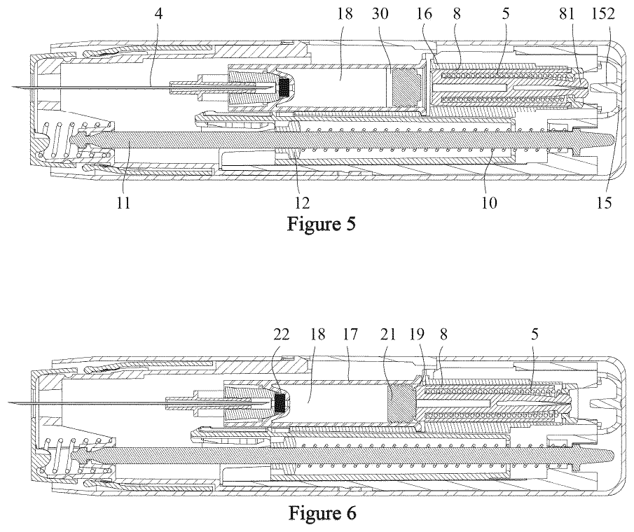

FIG. 5 is a cross-section of the drug delivery device of FIG. 1 whereby the spring boss 12 has been displaced in the proximal direction under the force of the needle insertion spring 10 along the spring shaft 11. The proximal displacement of the drug container holder 16 has removed the release pin protrusion 152 of the release pin 15 from adjacent to the distal plunger rod arms 81 as previously described in relation to FIGS. 4c and 4d.

The distal plunger rod arms 81 and release pin protrusion 152 may be designed such that the first stored energy source 5 is released at a desired point, for example at a specified time after the activation means has moved to the second position or after the plunger rod 8 has moved a specified distance relative to the release pin protrusion 152. For example, the proximal length of the release pin protrusion 152 may be shortened to release the plunger rod 8 sooner.

In the embodiment shown in FIG. 5, the first stored energy source 5 is released such that the force is applied to the drug 18 at around 12 ms before the needle 4 reaches the needle insertion position, and at the point when the needle 4 has travelled around 10% of the distance between the needle pre-insertion position and the needle insertion position.

FIG. 6 is a cross-section of the device of FIG. 1 where the first stored energy source 5 has expanded to move the plunger rod 8 in the proximal direction relative to the drug container assembly. The plunger rod 8 has been displaced such that it has pierced and unsealed the opening seal 19 on the distal opening of the drug container 17. After piercing the opening seal 19, the plunger rod 8 abuts the impacting surface of the piston 21 located between the drug 18 and distal opening of the drug container 17. At the point of impact, the momentum difference between the plunger rod 8 and the piston 21 is approximately 0.02 kgms⁻¹. The momentum difference may be attributed to the impacting components colliding at a relative velocity of approximately 13 ms⁻¹. The abutment, or impact, of the proximal face of the plunger rod 8 and the impacting surface of the piston 21 results in the force being applied to the drug 18. This force may mix the drug 18. Without being bound by theory, it is believed that mixing may be aided, under certain circumstances, by air in the drug container 17. In the pre-activation position, the drug container 17 contains an air bubble 30 having a volume of around 120 mm³ (at around atmospheric pressure and a temperature of around 20 degrees Celsius). Mixing may be aided by rapid compression of air located at a distal end of the drug container 17. After being rapidly compressed, this air may ‘explode’ into multiple bubbles which travel through the drug 18 and help to mix the drug 18. The rubber plug 22 remains coupled to the drug container 17, continuing to seal the proximal outlet at this stage.

FIG. 7 shows the device after the needle insertion spring 10 has expanded further to further displace the spring boss 12 along the spring shaft 11 and locate the needle 4 in the needle insertion position. In this position, a proximal face of the needle holder 26 abuts a surface of the front chassis 14. When the needle holder 26 abuts the front chassis 14, the needle 4 is in the needle insertion position and the abutment of the needle holder 26 against the front chassis 14 prevents the needle 4 from moving proximally beyond the needle insertion position. However, the spring boss 12 is configured such that when the needle 4 is in the needle insertion position, there remains further travel of the spring boss 12 along the spring shaft 11 under the action of further expansion of the needle insertion spring 10. As the spring boss 12 travels this distance, the drug container assembly is moved in the proximal direction relative to the needle 4 and needle holder 26 as shown in FIG. 8.

As shown in FIG. 8, the needle holder 26 travels within an inner cavity of the sealing retainer 23 until a distal face of the needle holder 26 abuts an inner surface of the sealing retainer 23. During this movement, the distal end of the needle 4 pierces the rubber plug 22. In this manner, the proximal outlet of the drug container 17 is unsealed for the drug 18 to be delivered through, after the needle 4 has reached the needle insertion position. In this embodiment, the proximal outlet of the drug container 17 is unsealed approximately 20 ms after movement of the activation means from the first position to the second position and approximately 12 ms after the force is applied to the drug. Once this outlet is unsealed, the piston 21 is permitted to travel relative to the drug container 17 to deliver the drug 18 through the needle 4 under the biasing of the first stored energy source 5. In this embodiment, the outlet is unsealed after the needle 4 has reached the needle insertion position.

FIG. 9 shows the device after the piston 21 has travelled proximally through the drug container 17 to deliver the drug through the needle 4. The needle 4 can then be removed from the injection site by pulling the device away from the injection site. Alternatively, a needle retraction mechanism could be incorporated into the device to withdraw the needle into the device following completion of drug delivery.

Thus, through the description of the sequence of operation of the drug delivery device, it is clear that a method of mixing a drug in a drug delivery device is provided. The device includes a drug dispensing mechanism comprising the first stored energy source 5 and the piston 21. The method of mixing the drug comprises applying the force to the drug using the first stored energy source 5. This mixes and increases a homogeneity of the drug 18 in the drug container 17, and also reduces a dynamic viscosity of the drug 18.

1. A drug delivery device comprising:
a housing portion;
a drug container assembly including a drug container containing a drug;
a needle through which the drug is dispensed in use, the needle being moveable relative to the housing portion from a needle pre-insertion position to a needle insertion position;
a plunger rod spaced from the drug container assembly prior to movement of the needle from the needle pre-insertion position;
a first stored energy source;
a needle insertion stored energy source; and
an activation means;
wherein the drug container comprises an outlet through which the drug is dispensed in use, the outlet being sealed prior to movement of the needle from the needle pre-insertion position, and
wherein the device is configured such that movement of the activation means from a first position to a second position releases the needle insertion stored energy source to move the needle from the needle pre-insertion position to the needle insertion position and releases the first stored energy source to move the plunger rod towards and relative to the drug container assembly such that the drug container assembly applies a force to the drug before the needle reaches the needle insertion position.

2. The drug delivery device according to claim 1, wherein the drug container assembly applies the force to the drug after the needle has moved from the needle pre-insertion position.

3. The drug delivery device according to claim 1 or 2, wherein movement of the activation means from the first position to the second position causes the first stored energy source to move the plunger rod towards the drug container assembly such that the plunger rod or an impacting component impacts an impact surface of the drug container assembly such that the drug container assembly applies the force to the drug.

4. (canceled)

5. The drug delivery device according to claim 3, wherein, following movement of the activation means from the first position to the second position, the plunger rod or the impacting component impacts the impact surface with a momentum difference between the plunger rod or the impacting component and the impact surface of at least 0.01 kgms⁻¹.

6. (canceled)

7. The drug delivery device according to claim 3, wherein the drug container assembly comprises a piston, the impact surface is a surface of the piston, and, in use, the piston moves relative to the drug container to dispense the drug through the needle.

8. (canceled)

9. (canceled)

10. The drug delivery device according to claim 7, wherein the drug container comprises, in addition to the outlet, an opening sealed by an opening seal prior to movement of the needle from the needle pre-insertion position and wherein, prior to movement of the needle from the needle pre-insertion position, the piston is located within the drug container and between the drug and the opening seal.

11. The drug delivery device according to claim 10, wherein movement of the activation means from the first position to the second position causes the first stored energy source to move the plunger rod towards the piston so as to pierce the opening seal.

12. The drug delivery device according to claim 1, wherein the drug container contains a gas bubble in addition to the drug.

13. The drug delivery device according to claim 12, wherein, prior to movement of the activation means from the first position to the second position, the gas bubble occupies at least 5% of he a volume enclosed by the piston and drug container at room temperature and atmospheric pressure.

14. The drug delivery device according to claim 1, wherein the device comprises an unsealing mechanism for unsealing the outlet.

15. The drug delivery device according to claim 14, wherein the unsealing mechanism is configured to unseal the outlet after the force is applied to the drug.

16. The drug delivery device according to claim 14, wherein the unsealing mechanism is configured to unseal the outlet after the needle has reached the needle insertion position.

17. The drug delivery device according to claim 14, wherein the unsealing mechanism is configured to unseal the outlet at least 5 ms after the force is applied to the drug.

18. The drug delivery device according to claim 1, wherein the drug container assembly applies the force to the drug at least 5 ms before the needle reaches the needle insertion position.

19. The drug delivery device according to claim 1, wherein the first stored energy source is released to move the plunger rod towards and relative to the drug container assembly after movement of the needle from the needle pre-insertion position but before the needle reaches the needle insertion position.

20. The drug delivery device according to claim 1, wherein the device comprises a housing and the activation means comprises a skin contact element movable relative to the housing, and wherein movement of the activation means from the first position to the second position comprises a distal movement of the skin contact element relative to the housing.

21. The drug delivery device according to claim 1, wherein the drug comprises one or more of: a non-Newtonian fluid, a non-Newtonian gel, a solute dispersed in a solvent, and/or wherein the drug is not homogeneous in its lowest energy state and/or wherein, in use, the force applied to the drug reduces a dynamic viscosity of the drug.

22. (canceled)

23. The drug delivery device according to claim 1, wherein the device is an autoinjector.

24. A method of mixing a drug in a drug delivery device, the device comprising:
a drug dispensing mechanism including a stored energy source; and a drug container containing a drug, and the method comprising:
applying a force to the drug using the stored energy source to mix and increase a homogeneity of the drug in the drug container and/or to reduce a dynamic viscosity of the drug in the drug container.

25. The method according to claim 24, wherein applying the force to the drug comprises impacting a plunger rod or an impacting component against an impact surface.
